(19) United States
(12) Patent Application Publication
(10) Pub. No.: US 2025/0147023 A1
(43) Pub. Date: May 8, 2025

(54) RAPID DETECTION OF AT LEAST ONE TOXIN USING A MICROFLUIDIC DEVICE

(71) Applicant: BLUE LION LABS LTD., Kitchener (CA)

(72) Inventors: Jason DEGLINT, Kitchener (CA); Mahla POUDINEH, Kitchener (CA); Kathryn THOMAS, Kitchener (CA); Hamid AGHAMOHAMMADI, Kitchener (CA)

(21) Appl. No.: 18/729,924

(22) PCT Filed: Jan. 25, 2023
(86) PCT No.: PCT/CA2023/050088

§ 371 Date: Jul. 18, 2024

Related U.S. Application Data
(60) Provisional application No. 63/302,682, filed on Jan. 25, 2022.

(51) Int. Cl. G01N 33/569; G01N 15/10; G01N 15/1434; G01N 33/543; G01N 33/58

(52) U.S. Cl. CPC G01N 33/56911; G01N 15/1434; G01N 33/54326; G01N 33/588; G01N 2015/1006; G01N 2333/195; G01N 2333/765

(57) ABSTRACT

The disclosure is directed at a method and system for detecting at least one toxin in a sample using a microfluidic device. The sample is mixed with a reagent within a mixing module to generate a mixture solution. The mixture solution is then collected in a detection chamber and tested for its fluorescence. The reagent being used includes beads that have been functionalized with the toxin of interest and BSA molecules.

### CROSS-REFERENCE TO OTHER APPLICATIONS

This application claims priority from U.S. Provisional Application No. 63/302,682 filed Jan. 25, 2022 which is hereby incorporated by reference.

### FIELD

The disclosure is generally directed at toxin detection, and more specifically, at a method and system for rapid detection of at least one toxin in a microfluidic device.

### BACKGROUND

Harmful algae blooms (HABs) occur when colonies of algae, such as cyanobacteria, grow out of control. These gram-negative bacteria produce toxins, such as hepatotoxins and neurotoxins, that can cause a wide range of adverse human health effects including, but not limited to, neurological, gastrointestinal, and/or skin disorders. The toxins are a group of biochemical contaminants existing in the environment that can significantly harm animal and human health. Examples include microcystin (MCs) and cylindrospermopsin (CYN).

Among MCs, microcystin-leucine-arginine (MC-LR) is considered the most common and toxic because it is a potential hepatotoxin, neurotoxin, and tumor promoter. Accordingly, a maximum concentration of 1 μg/L for MC-LR and CYN in drinking water has been established to reduce health risks for humans. Despite the measures taken, the normal occurrence of HABs, particularly cyanobacteria, is worsening because of anthropogenic activities, such as agricultural run-off, urban waste, manufacturing of detergents, the release of excess amounts of carbon dioxide, and/or global warming.

Traditional techniques for MC-LR detection primarily use high-performance liquid chromatography (HPLC) combined with either an ultraviolet-visible detector or mass spectrometry (MS) and enzyme-linked immunosorbent assay (ELISA). Although these techniques are highly sensitive and specific, the high cost of equipment and tests, requirement for skilled personnel, long-time needed for sample pre-treatment and analysis greatly hinder their application for in-field detection of toxins. ELISA techniques provide a simple method for screening MCs but suffer from low sensitivity and lack of specificity compared with the instrumental assays.

Therefore, there is provided a novel system and method for rapid detection of at least one toxin in a microfluidic device.

### SUMMARY

The current disclosure is directed at a novel system and method for rapid detection of toxins. In one embodiment, the disclosure includes a microfluidic device that includes a mixing module for mixing a sample and a reagent. The mixed solution is then collected in a detection chamber and measured to determine if a toxin is present in the sample.

The use of microfluidics and lab-on-a-chip platforms for toxin detection provides numerous advantages, including, but not limited to, at least one of: reduced consumption of expensive reagents, rapid isolation with high quality and high throughput, and/or demonstrating a cost-effective platform for detecting toxins. From an operational standpoint, microfluidic devices are automated, portable, and user-friendly. These devices integrate all conventional analysis steps and are promising for developing a commercialized toxin detection sensor.

In one embodiment, the disclosure is directed at the integration of a mixing module and detection chamber for providing a multiplex detection platform. In another aspect, the disclosure is directed at the production of a bead-based immunoassay for the detection of toxins. In another aspect, the disclosure is directed at a microfluidic device for multiplex detection of small molecules in low concentration which overcomes current regular detection techniques.

In an aspect of the disclosure, there is provided a method of rapid detection of at least one toxin in a sample using a microfluidic device including mixing the sample with a selected reagent having metallic beads with functionalized toxins attached to a surface of the metallic beads to generate a mixture solution; incubating the mixture solution; and measuring a fluorescence signal of the beads.

In another aspect, incubating the mixture solution includes incubating the mixture solution with polyclonal antibodies with quantum dots (pAb-QDots) to provide fluorescence to the beads. In a further aspect, the method includes washing the beads after incubating the mixture solution. In yet another aspect, the method includes, before mixing the sample, dividing the sample into at least two streams. In a further aspect, mixing the sample with a selected reagent includes mixing each of the at least two streams with a different reagent to generate different mixture solutions, each of the different reagents including metallic beads with functionalized toxins attached to a surface of the metallic beads. In another aspect, measuring a fluorescence signal of the beads is performed by flow cytometry, a fluorescence microscope or a plate reader. In an aspect, the method further includes transmitting the fluorescence signal to a display.

In another aspect of the disclosure, there is provided a system for rapid detection of at least one toxin in a sample including a sample receiving area for receiving the sample; at least one reagent area for receiving a reagent for detection of the at least one toxin in the sample, the reagent including metallic beads with functionalized toxins attached to a surface of the metallic beads; and at least one detection array in fluid communication with the at least one reagent area and the sample receiving area to generate a mixture solution of the sample; wherein the at least one detection array includes a mixing module including a herringbone structure.

In another aspect, the at least one detection array further includes a detection chamber for receiving the mixture sample. In yet another aspect, the system includes a fluorescence reader. In a further aspect, the fluorescence reader is a flow cytometry apparatus, a fluorescence microscope or a plate reader. In yet a further aspect, the system includes a flow control apparatus for controlling flow rates of the sample and the reagent. In another further aspect, the sample receiving area further includes an inlet for receiving the sample. In another aspect, the system includes an apparatus for collecting the beads within the detection chamber.

In yet a further aspect of the disclosure, there is provided a method of reagent production for detection of a toxin of interest in a sample including combining the toxin of interest with bovine serum albumin (BSA) molecules; functionalizing the combination of the toxin of interest and the BSA molecules to a surface of a set of metallic beads; and mixing functionalized metallic beads with an antibody.

In another aspect, functionalizing the combination is performed by EDC or NHS chemistry.

### DESCRIPTION OF THE DRAWINGS

Embodiments of the present disclosure will now be described, by way of example only, with reference to the attached Figures.

FIG. 1a is a schematic diagram of a toxin detection environment including a microfluidic device for rapid detection of at least one toxin;

FIG. 1b is a photograph of an embodiment of the microfluidic device of FIG. 1a;

FIG. 1c is a set of fluorescence images showing beads retained inside a detection chamber;

FIG. 1d is a microscopic image of a herringbone structured serpentine channel embedded in the mixing microfluidic device;

FIG. 2a is a flowchart showing a method of rapid detection of at least one toxin;

FIGS. 2b to 2d are schematic diagrams of an indirect competitive assay for detection of MC-LR;

FIGS. 3a and 3b are fluorescence images of beads after a benchtop indirect competitive assay;

FIGS. 3c and 3d are charts showing optimization experiment results using a toxin monoclonal capture antibody;

FIGS. 3e and 3f are graphs showing fluorescence signals vs toxin concentration for a standard benchtop experiment;

FIG. 3g is a set of fluorescence images in a detection chamber;

FIG. 4 is a graph showing a comparison of mean fluorescence intensity vs fluid flow rate for different mixing modules;

FIG. 5a is a microscopic image of a detection chamber;

FIG. 5b is a chart showing a comparison or bead retention in the detection chamber for different flow rates;

FIG. 5c are a set of microscopic images of retained fluorescence magnetic beads inside the channel for different flow rates;

FIGS. 6a and 6b are fluorescence images of beads before (FIG. 6a) and after (FIG. 6b) incubation;

FIG. 7 is a set of fluorescence images of beads in a detection chamber;

FIGS. 8a and 8b are graphs showing calibration curves that correlate fluorescence signal intensity with toxin concentration for MC-LR (FIG. 8a) and OA (FIG. 8b) in microfluidic detection platform;

FIGS. 8c and 8d are graphs showing performance of a MC-LR assay (FIG. 8c) and an OA assay (FIG. 8d) in the presence of interference, CYN and STX with concentration of 1 μg/ml; and

FIG. 9 is a set of images showing fluorescence beads in a detection chamber over a three week period.

### DETAILED DESCRIPTION

The current disclosure is directed at a system and method for rapid detection of at least one toxin using a microfluidic device. In one embodiment, the disclosure may be seen as a platform, which may be referred to as a toxin-chip, for rapid, multiplexed detection of at least one toxin using a microfluidic device. Using the system and method of the disclosure, rapid detection of at least one toxin in a liquid sample may be performed. In one specific embodiment, the disclosure is directed at a novel, rapid, scalable, and cost-effective approach to detect threatening toxins in a water sample.

In one embodiment, the disclosure is directed at the integration of microfluidic advancements with optical measurement for simultaneous detection of toxins, such as, but not limited to, MC-LR and CYN in a liquid sample. This may be performed with high sensitivity and specificity. As discussed below, experiments using the system and method of the disclosure were performed to determine the performance of embodiments of the disclosure with both known concentrations of toxins and unknown concentrations of toxins in real environmental water samples.

In experiments, under preferred, or optimal, conditions, the system and method of the disclosure quantitatively analyzed the presence of MC-LR and other toxins (such as CYN) with a detection limit that satisfies the regulatory guidelines for drinking water, thus providing a powerful and innovative tool for rapid and sensitive monitoring of at least one toxin in environmental samples using a microfluidic device. While experiments were performed under controlled conditions, the system and method of the disclosure may also be used in other, non-optimal conditions and for outside drinking water (e.g. other freshwater uses and in marine environments). In a further embodiment, the system and method of the disclosure may be modified for rapid detection of other toxins outside of MC-LR and CYN.

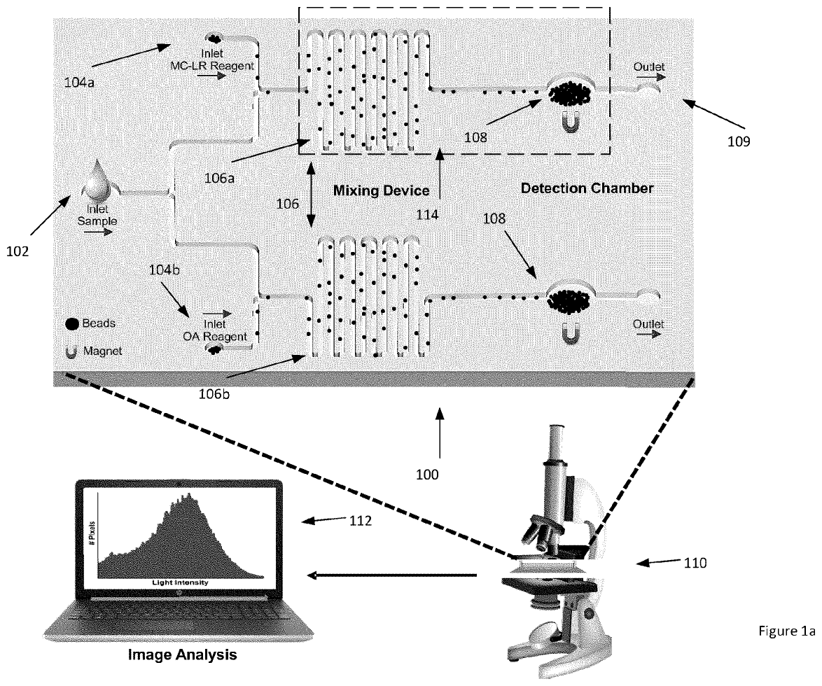

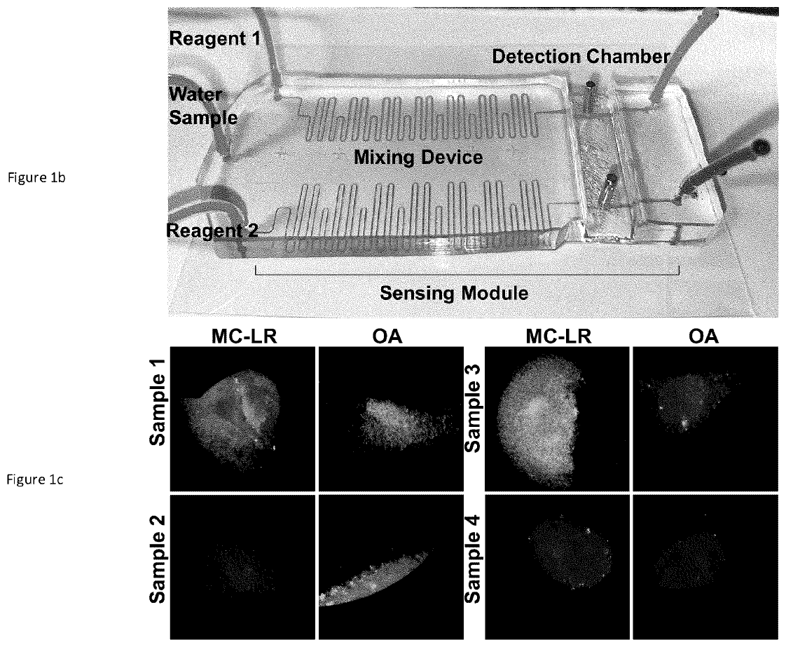

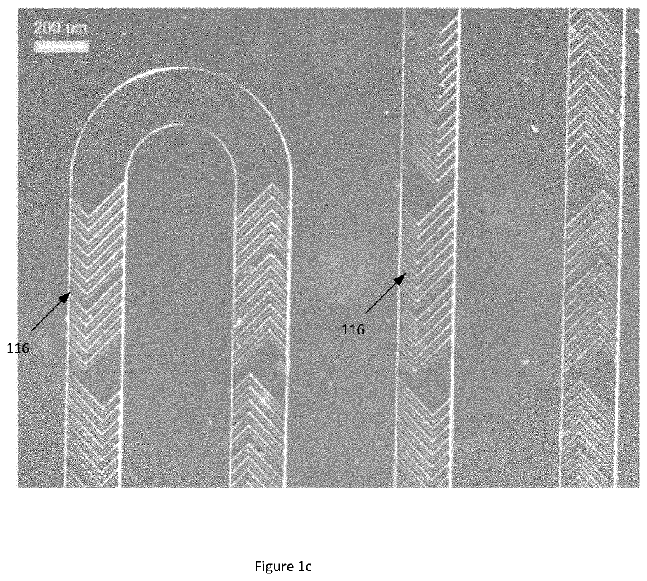

Turning to FIG. 1a, a schematic diagram of a toxin detection environment including a microfluidic device for rapid detection of at least one toxin is shown. In one embodiment, the disclosure integrates an immunobead-based assay into a microfluidic device for simultaneous and multiplexed detection of at least one toxin.

The system, or device, 100 (which may be referred to as a device platform, a microfluidic device or a toxin detection device) includes a sample receiving area 102 (or sample inlet), for receiving a liquid sample, such as, but not limited to, water. The sample inlet may include a tube portion for receiving a continuous flow of the liquid sample or for receiving an input of the liquid sample that is to be stored in the sample receiving area. The device 100 may also include pumps for moving the liquid sample through the device if the liquid sample is not being injected into the device. A photograph of one embodiment of the device 100 is shown in FIG. 1b.

The device further includes a set of toxin reagent areas 104 (or toxin inlets). The toxin reagent inlets may include a tube portion for receiving a continuous flow of a toxin reagent or for receiving an input of the toxin reagent that is to be stored in the toxin reagent area. The tube portion may be connected to a pump that continuously pumps or injects the reagent into the device. In another embodiment, the reagent may be freeze dried in the toxin reagent areas 104. The toxin reagent area 104 may be designed in a similar fashion with respect to the sample receiving area 102. In the current embodiment, there are two toxin reagent areas 104a and 104b for receiving toxin reagent solutions. As will be understood, there can be any number of toxin reagent areas 104 depending on a size and/or design of the device 100. Each toxin reagent area receives a reagent that is used to detect a specific toxin within the liquid sample. In the current embodiment, one of the two toxin reagent areas may be for receiving a MC-LR reagent (area 104a) and the other toxin reagent area may be for receiving an okadaic acid (OA) reagent (area 104b). In another embodiment, one of the two toxin reagent areas may be for receiving a MC-LR reagent and the other toxin reagent area may be for a CYN reagent. As will be discussed in more detail below, each of the reagents includes a set of beads within the reagent. In one embodiment, the beads are metallic beads coated with the toxin of interest or that is to be detected. In some embodiments, in order to provide a reagent that is more directed towards the toxin of interest, the beads may be functionalized with the toxin on a surface of the bead prior to its injection into the device.

The system 100 further includes a chaotic mixing, or reaction module, or area 106, where the liquid sample mixes with the reagent from one of the different toxin reagent areas 104. In the current embodiment, there are two reaction areas 106a and 106b, each associated with one of the toxin reagent areas 104. Although shown in a serpentine shape, the mixing module 106 may be any shape or straight.

The reaction area 106 is in fluid communication with the sample receiving area 102 and toxin reagent area 104 via channels that are etched or integrated within the device 100. In the current embodiment, there is one reaction area 106 associated with or for each of the toxin reagent areas 104. In one embodiment, the channels within the reaction area 106 include a physical herringbone structure in order to improve the level of mixing between the liquid sample and the reagent. This is schematically shown in FIG. 1d and discussed below. The target toxins (within the liquid sample) are mixed with the reagent solutions in the individual reaction areas 106 whereby mixing of the liquid sample and the reagent results in a biological reaction and attachment of fluorescence biomolecules to the beads to become fluorescent beads.

Each reaction area 106 is in fluid connection (such as via a channel) with a detection chamber 108 to pass the mixture solution. In each detection chamber 108, the fluorescent beads are collected for fluorescence labeling and measurement. In the current embodiment, the system includes a magnet that draws the beads to one side of the detection chamber and create a pallet for capturing fluorescence images, such as via a microscope 110. In other embodiments, the fluorescent beads may be removed from the detection chamber 108 (via an outlet 109) for examination. The images captured by the microscope 110 may be transmitted to a display 112 for image analysis.

In some embodiments, the combination of a reaction area 106 and a detection chamber 108 may be seen as a detection array 114 for the associated reagent area 104. In other embodiments, the combination of a reagent area 104, a reaction area 106 and a detection chamber 108 may be seen as the detection array 114 whereby a device 100 may be seen as including a set of detection arrays 114. In some embodiments, there may only be a single toxin reagent area 104, one mixing module 106 with a physical herringbone structure and one detector chamber.

In the current embodiment, as the liquid sample passes from the sample receiving area 102 to the individual mixing modules 106, the sample is divided into two streams as the liquid sample travels within the channels of the device 100. The flow rate of the liquid sample may be controlled by an individual or apparatus injecting the liquid sample into the device 100 or may be controlled via a flow rate controller apparatus associated within the device. In some embodiments, the apparatus injecting the liquid sample may be controlled by a flow controller. Division of the sample may be due to a design of the channels within the device connecting the sample receiving area 102 and the mixing module 106.

In other embodiments, the sample may be divided into any number of streams (based on a design of the device 100) depending on predetermined criteria such as, but not limited to, the number of detection arrays 114 or the number of toxins being detected. In some embodiments, not all of the streams may be used. For example, in some embodiments, there may be five toxin reagent areas but only three are being used such that the sample is divided into the five streams but the streams only mix with the reagents in the three mixing areas associated with the three toxin reagent areas receiving reagent.

In one embodiment of operation, each divided stream is directed to a separate detection array 114 associated with one of the toxin reagent areas 104. As discussed above, each detection array 114 may include an individual mixing module 106 and detection chamber 108 for a specific toxin (or toxin reagent area) 104. As will be understood, selection of the reagent that is placed within a reagent area 104 varies for each detection array 114 and is selected for a specific or target toxin or toxins that may be located within the liquid sample received in the sample receiving area 102. In another embodiment, the immunoassay utilizes antibodies as a recognition element that is selective toward the target toxin. Therefore, the target toxin is selectively measured among interferences, and any possible cross-reactivity is avoided.

Turning to FIG. 1d, an enlarged view of a portion of a mixing module is shown. FIG. 1d provides a microscopic image of a herringbone structured channel embedded in the mixing module. The herringbone structure is defined by grooves 116. The left side of FIG. 1d shows a serpentine shape channel with herringbone grooves while the right side of FIG. 1d shows two different straight portions of a channel with herringbone grooves.

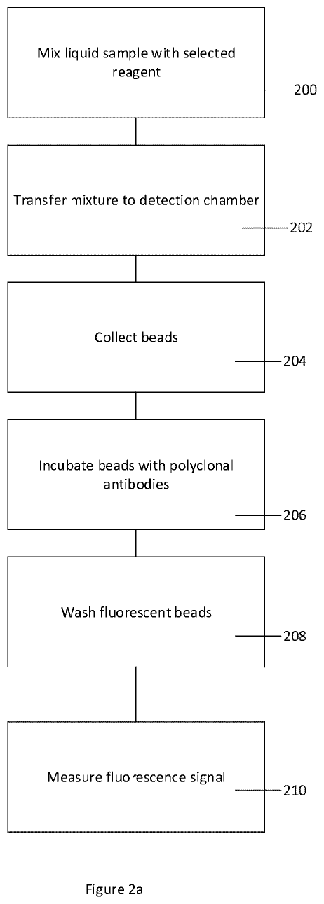

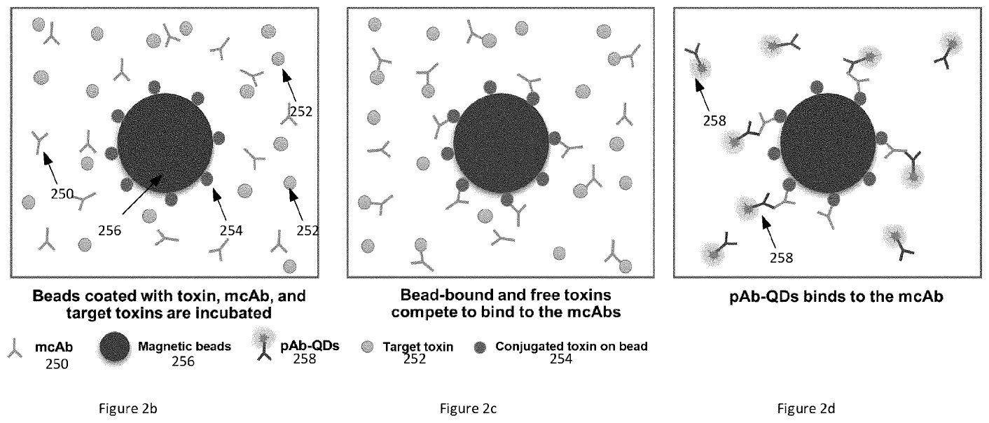

Turning to FIG. 2a, a flowchart showing one embodiment of a method for rapid detection of at least one toxin in a microfluidic device is shown. In some embodiments, the reagent may be manually prepared prior to use. In one embodiment of reagent preparation, the metallic beads may have functionalized toxins applied to a surface of the beads. In one embodiment of reagent preparation, the target toxin of interest is combined with bovine serum albumin (BSA) molecules (such as, but not limited to, MC-LR-BSA) which are then functionalized on the surface of the metallic beads. This may be achieved via through EDC or NHS chemistry although other surface functionalization chemistries are contemplated. The metallic beads are then mixed with an antibody prior to injection or insertion into the device. In other embodiments, previously prepared reagents may be used.

In one embodiment, the method may be seen as a modified version of an indirect competitive ELISA toxin detection assay. FIGS. 2b to 2d are schematic diagrams showing an indirect competitive assay for detection of MC-LR,

In order to test the device of the disclosure, a sample of water was tested using the device of FIG. 1a where MC-LR reagent was placed/injected into one of the reagent areas 104a and a OA reagent was placed in the other of the reagent areas 104b. After the sample and the reagents are placed in their respective locations, the method of rapid detection of at least one toxin may start. In another embodiment, the sample and the reagents may be continuously injected/pumped into the sample 102 and reagent areas 104, respectively to provide a continuous flow of the liquid sample and the reagent. In some embodiment, the reagents may be manually prepared prior to injection into the device.

Initially, the liquid sample and the reagents flow from their respective starting areas along the channels towards a mixing module associated with the reagent where they are able to mix together or to be incubated together (200). As discussed above, the flow rate may be controlled via a flow control apparatus or may be controlled by being continuously injected into the device. As schematically shown in FIG. 2b (during initial mixture), a toxin monoclonal antibody 250, free target toxin 252, and the toxin functionalized on a magnetic bead 254 and a magnetic bead 256 are mixed together to create the mixture solution.

Within the mixing module, the competition happens between the free toxin 252 and surface-bound toxin 254 to occupy antibody binding sites such as schematically shown in FIG. 2c. The resulting mixture or mixture solution is then passed to the detection chamber (202) where the beads are then collected or positioned (204). In one embodiment, the collection of the beads may include using a magnet to position or attract the metallic beads against one wall of the detection chamber. In another embodiment, the collection of the beads may be via an apparatus that can position the beads in a predetermined position within the detection chamber for examination by a fluorescence reader. In yet another embodiment, collecting the beads may include retrieving the beads from an outlet and then placing the beads within a well for examination by a card reader, and the like.

The fluorescent beads are then incubated or mixed with polyclonal antibodies with quantum dots (pAb-QDots) or any type of fluorophore (206). In one embodiment, the pAb-QDots, or detection antibodies with a fluorescence tag, are then added to the solution to serve as detection probes. The pAb-QDot binds to the MC-LR antibody captured on the bead's surface and produces a measurable fluorescence signal or detection antibody with fluorescence tag 258 as shown schematically in FIG. 2c such that the beads may be seen as fluorescent beads. In another embodiment, the magnetic beads are functionalized with the MC-LR BSA which is a carrier protein that helps with MC-LR surface functionalization and reactivity with free MC-LR monoclonal antibody without interfering with the immunoreaction.

The fluorescent beads are washed (208) to remove excess fluorophore and other materials from a surface of the beads which may affect the fluorescence signal being detected. The fluorescence signal of the beads is then measured (210). This may be performed in different ways. In one embodiment, the measurement of the fluorescence signal of the beads may be performed via a microscope. In another embodiment, the beads may be collected from an outlet and transferred to a set of well plates and the fluorescence signal read by a plate reader. The results may then be displayed on the display.

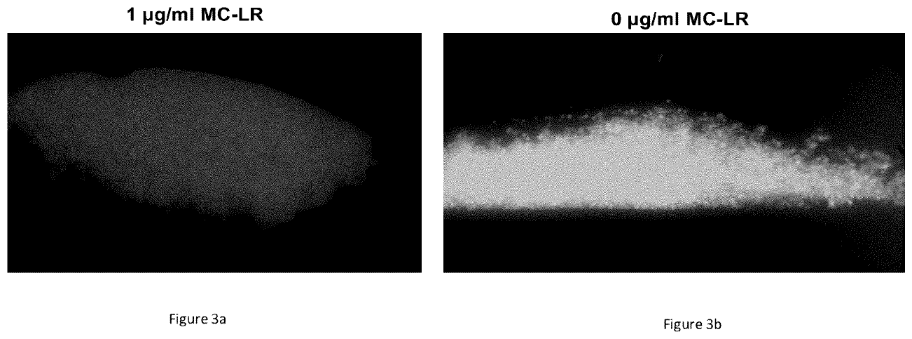

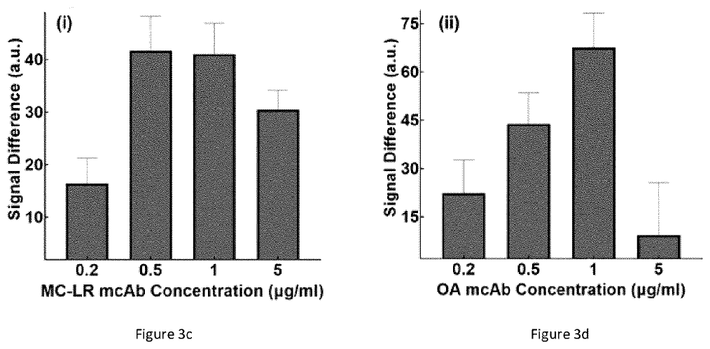

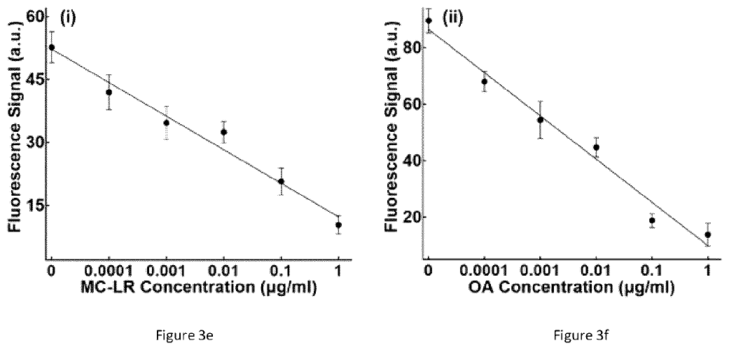

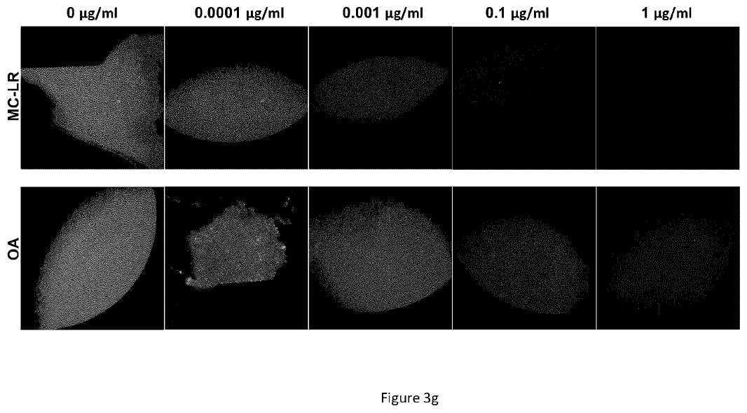

In order to validate the method of the disclosure, microscopic images of samples were compared. In one embodiment, the beads, or microbeads, flowed into the detection chamber where an external magnetic force was applied to retain the bead. The fluorescence images, such as shown in FIGS. 3a and 3b, showed a decrease in the fluorescence signal of beads incubated with higher MC-LR concentrations when using the device of the disclosure.

In another embodiment of reagent production, the mixture solution included a toxin monoclonal capture antibody (mcAb), the target toxin, and magnetic beads functionalized with a BSA-toxin. Different concentrations of mcAb were examined or studied to determine a preferred or optimal concentration for the competitive assay or reagent solution. The results are shown in FIGS. 3c and 3d. Low concentrations of mcAb resulted in an indistinguishable signal for higher concentrations of toxin due to a lack of mcAbs for the targets in the solution. On the other hand, high mcAb concentrations caused saturation at the lower concentrations of the target toxin as the abundance of mcAb provided sufficient antibodies to bind to the bead-bound toxin regardless of the target toxin concentration.

Reagent solutions were tested using different mcAb concentrations and the fluorescence signal difference between the highest and lowest concentration of the target toxin were investigated. As shown in FIG. 3c, it was determined that approximately 0.5 to 1 μg/ml of capture antibody was one optimal concentration for MC-LR mcAb and OA mcAb (in a reagent solution).

Validation of the performance of the bead-based, indirect competitive assay of the disclosure was performed with a benchtop experiment with a reagent having an optimized mcAb concentration (as outlined above). The performance of the reagent for detection was verified by analyzing different concentrations of two target toxins (MC-LR and OA) in a range of 0 to 1 μg/ml. The fluorescence images (as schematically shown in FIG. 3g) and measured fluorescence signals (FIGS. 3e and 3f) showed a decrease in the fluorescence signal intensity of beads incubated with higher concentrations of toxin.

With respect to the fabrication of a microfluidic device 100 for rapid detection of toxins, such as the one schematically shown in FIG. 1a, each component of the detection device 100 may be fabricated individually to improve and/or optimize device functionality. It is understood that some components may be fabricated together.

For the mixing module 106 of the device 100, in one embodiment, where the sample and reagent solutions are injected through inlets of the device (such as in the embodiment of FIG. 1b), the liquid sample and reagent solution are efficiently mixed in the herringbone structured channel or reaction area 106. While a conventional benchtop assay requires a long incubation time (hours) because of the inefficient mass transport of the analyte to the surface of beads, in the system of the disclosure, the herringbone structure of the mixing module induces a chaotic mixing between the sample and the reagent and enhances a rate of molecular diffusion thereby reducing the incubation time, in some embodiments to less than a minute. The mixing module 106, fabricated with an improved or optimized geometry and herringbone structures incorporated inside the channel, achieves rapid and continuous mixing of reagents and samples within the microfluidic channels of the mixing module.

In another specific embodiment of a detection device 100, the mixing modules may include a herringbone structured channel having a total length of approximately 35 cm but may be between 20 to 40 cm depending on an application or purpose of the channel. The channel may also have a cross-section of approximately 300 μm×approximately 45 μm. In other embodiments, the cross-section may be between (approximately 50 to approximately 1000 μm)×(approximately 20 to approximately 500 μm). In another embodiment, the channel of the mixing area is serpentine in shape to fit a long incubation channel in the dimensions of regular glass slides. Moreover, the radius of the turns in the serpentine shaped channel can be selected such that they are large enough so the effects of these turns can be neglected.

In experimental testing, a mixing module channel including herringbone structures with a height of approximately 45 μm was tested against a simple (without a herringbone structure) mixing module channel. Both devices (simple and herringbone structured) had two inlets and one outlet (or detection chamber) where the beads were collected. Using a standard Immunoglobulin (IgG) immunobead-based sandwich, ELISA was performed using both devices to compare the efficiency of the two devices.

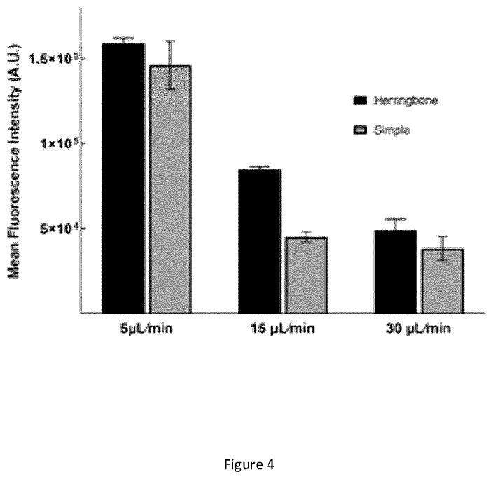

A reagent sample including microbeads (or beads) functionalized with IgG capture antibody (cAb) (˜10⁵ bead/ml) and fluorescently tagged detection antibodies (dAb) (10 μg/ml) was introduced through the reagent inlet 104 while a buffer solution (liquid sample) containing IgG (1 μg/ml) was injected via the sample inlet 102 into the device 100. These solutions were injected at different flow rates (5, 15, 30 μL/min) with a syringe pump. The beads were collected from the outlet and their fluorescence signal measured using flow cytometry. The results are shown in FIG. 4 with the readings from the mixing module with the herringbone structure in black and the simple mixing module in grey.

Comparing the fluorescence signal intensity obtained at different flow rates using the simple and herringbone channels, the results showed that herringbone structure was more effective to capture IgG. Specifically, at the flow rate of 15 μL/min, capture efficiency was enhanced by 83% which is higher than other flow rates. This is due to a trade-off between the bead's residual time and agitation inside the channel. At lower flow rates, residual time increases but the agitation is unlikely to happen whereas at higher flow rates, the flow is more agitated, and beads spend less time in the channel. As can be seen, there is an improvement in the detection using a mixing module with the herringbone structure within the channels of the mixing module.

With respect to the fabrication of a detection chamber 108, after mixing and reaction take place inside the mixing module 106, the magnetic beads enter the chamber where they are magnetically retained (such as via a magnet located within or on the detection chamber). The size of the magnet may match the size of the chamber. Experiments were performed, and different designs were tested to find an optimum, or preferred, detection chamber 108. In one embodiment, the detection chamber 108 is a circular chamber with a diameter of approximately 1.5 mm to 10 mm. With higher diametered detection chambers, further support to the channels may be integrated such as to provide further support to the walls of the channel such as to handle increased flow rate. Multiple experiments were carried out using this embodiment with respect to the beads' retention in order to capture a microscopic image from the pallet of beads in the detection chamber.

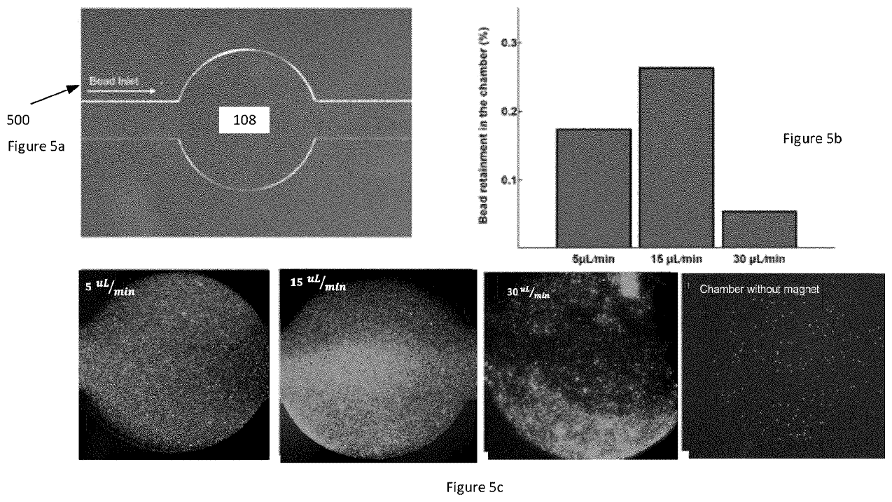

In one embodiment of testing the detection chamber 108, a bead (which may be fluorescent beads) solution is injected into the chamber using a syringe pump (in the direction of arrow 500) as schematically shown in FIG. 5a. The magnet was then placed on the outside of the chamber to retain the magnetic beads on an internal surface of the chamber. The detection chamber was then placed under the microscope to take images from retained beads. Different flow (or injection) rates were tested to improve or optimize the toxin detection process (from a flow rate standpoint) as outlined below with respect to the results shown in FIG. 5b.

In the chamber, the magnetic force from the magnet needs to overcome the drag force to retain the beads within the chamber. A lower flow rate (lower drag force) is easily prevailed by magnetic power and results in a higher retainment or retention ratio of the beads. On the other hand, a higher flow rate (higher drag force) avoids the settlement of beads in injection syringes, tubes, and channels and results in a higher number of beads entering the chamber. As results in FIG. 5b suggest, 15 ul/min is a preferred flow rate for beads retainment. The microscopic images of the chamber in FIG. 5c confirm that a flow rate of 15 ul/min yields a denser bead pallet for measurement. It is understood that other injection flow rates are considered.

With respect to the subject matter of the incubation of the beads with pAb-QDot, this may occur inside the detection chamber. The incubation may include the injection of detection reagents, which include a detection agent conjugated with a fluorophore, into the detection chamber to label the immunocomplex on the surface of beads for subsequent detection under a fluorescent microscope. In some embodiments, the detection chamber may include another inlet for receiving the detection reagent.

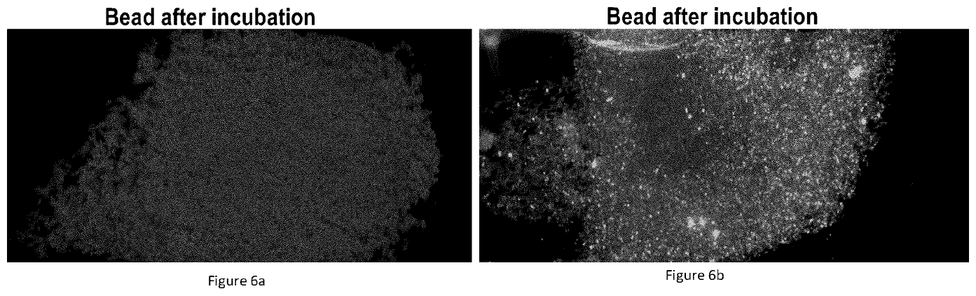

In experimentation, the pAb-QDot solution (5 ug/ml) was injected into the chamber to bind with the retained beads (˜10⁴ beads) in the detection chamber. Following incubation, the chamber was washed to remove the unbound pAb-QDot from the chamber. FIG. 6a shows is a fluorescence image of the retained beads before incubation while FIG. 6b is a fluorescence image of the retained beads after incubation. As can be seen, there is an observable difference between the fluorescence signals of beads in FIGS. 6a and 6b which confirm successful incubation.

In another embodiment of detection device manufacture, a standard microfluidic device fabrication protocol with glass substrates and polydimethylsiloxane (PDMS) may be used to build the devices. In one embodiment, three photomasks for a channel (preferably serpentine), a detection chamber, herringbone structures, and integrated device were designed and then printed.

The masks were utilized for the fabrication of master molds on clean silicon wafers. A layer of SU8-3050 was spin-coated on the wafer to form an approximately 45 μm thick layer. The wafer was then pre-baked at approximately 90° C., exposed to ultraviolet (UV) light with the serpentine channel photomask, and developed by submerging the device in SU-8 developer for 7 min. The single layered molds were hard baked at approximately 150° C. to finalize the fabrication of the initial serpentine channel. The other serpentine channel mold was post baked at approximately 90° C. to stabilize the channel structures, and then the second layer of SU8 was cast in the same manner. The herringbone mask was aligned with the serpentine channel structures on the mold with the help of a mask aligner. At last, the second layer was exposed and developed, completing fabrication of herringbone-structured serpentine channel.

Overnight salinization was performed on a fabricated mold in a vacuum desiccator. The PDMS and curing agent were mixed in a 10:1 ratio, and then the air bubbles were removed using a desiccator. The PDMS polymer was poured onto the molds and heated at approximately 70° C. in an oven for approximately 2 hours. The PDMS replicas were peeled off from the mold and cut into a predetermined shape. Inlets and outlet holes were punched in the PDMS mold for fluid injection. The PDMS structures and cleaned glass slides were then bound to each other with plasma treatment. The punched inlets and outlet were connected to silicone tubing to complete the microfluidic device fabrication. Before use, the devices were degassed with Pluronic™ solution overnight.

In order to address IgG capture inside the microfluidic device, in one experimental use, two immunoreactions took place inside the channels of the device and formed sandwich molecules on the bead. The reactions were between IgG and the cAb-conjugated beads and between dAb and the captured IgG. In experiments, both reactions took place simultaneously inside the mixing module. First, the biotinylated IgG cAbs were conjugated on the streptavidin-coated beads. The beads were then mixed with antibodies and placed in the shaker incubator for approximately 1.5 hours, allowing them to form biotin-streptavidin bond.

A syringe pump was used to inject the liquid sample and reagent solution into the device (for example, the device shown in FIG. 1a or FIG. 1b) through two inlets with a predetermined flow rate. The injected reagent included cAb conjugated beads (4×10⁵ beads/ml) and IgG dAb (10 μg/ml), and the injected liquid sample used was IgG (1 μg/ml). The two flows were mixed and reacted in the mixing module, and the beads were collected from the device outlet (or detection chamber). The fluorescence signal of the beads then was measured such as via flow cytometry, a fluorescence microscope or a plate reader.

In order to address pAb-QDot incubation inside the microfluidic device, a process was developed for the incubation of secondary antibodies with fluorescence tags inside the detection chamber. The magnetic beads were conjugated with MC-LR antibodies (target of pAb-QDot) and injected into the detection chamber. After bead pallet formation or collection by the magnet, the pAb-QDot solution was injected into the chamber at a reduced flow rate (15 ul/hr) and captured on retained beads. After a predetermined period of time, such as between 30 minutes and one hour of incubation, the chamber was washed by injecting a washing buffer with a flow rate of 15 ul/min for 15 minutes. During the washing step, the unbound pAb-QDot was removed from the chamber to avoid or reduce the presence of a background signal. The fluorescence images of the beads then were taken.

With respect to the use of flow cytometry to determine the fluorescence measurement, the beads collected from the device outlet or detection chamber were immediately centrifuged to avoid further incubation that can cause an error in the measurements. Two steps of washing were followed to prepare the beads for the flow cytometer measurements. The washed bead pellet was re-suspended in 200 μl of buffer solution and transferred to FACS™ tubes. Data was acquired and analysed and the gating of beads was performed based on FCS/SSC parameters so that unbound molecules or other possible aggregates are excluded from the analysis. The number and emitted fluorescence signal of gated beads was measured.

With respect to fluorescent microscope imaging, the fluorescence signal caused by the presence of fluorophore or quantum dots (QDots) on the beads was measured under the inverted microscope after sample preparations. The collected samples were injected into a circular chamber with an optimized flow rate. An external magnet was placed on top of the chamber to retain the magnetic beads. The bead's pallets were illuminated by a laser to capture the fluorescence tag (FITC or QDots) intensity which provides an indication of the amount of formed immunocomplex on the bead or beads.

With respect to the plate reader, after incubation, the beads are collected and washed. Then, the beads are re-suspended and transferred to a well plate. A fluorescence intensity of the beads within each well is then measured with a plate reader. Inside the plate reader, the quantum dots or FITC are excited in 420 nm or 495 nm and the emitted fluorescence signal measured in 605 nm or 519 nm.

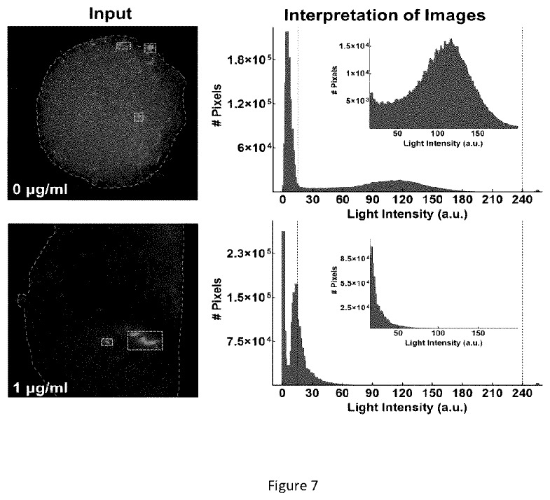

In another embodiment of the disclosure, the system may include an image recognition program or component that is used in conjunction with the device for detecting toxins. The image recognition component may be used to measure the fluorescence signal intensity and quantify the toxin concentrations by automatically calculating the concentration of the target toxin based on the fluorescence signal and embedded calibration functions. In training the image recognition component, many fluorescence images were utilized for training the program and determining an applicable light intensity range to extract the mean fluorescence intensity of the collected beads inside the detection chamber. This is schematically shown in FIG. 7. Threshold values were determined to exclude the background areas around the beads and the excessively bright fluorescence spots from the measurement as the excessively bright spots may be formed due to the agglomeration and entrapment of pAb-QDs that the washing step was not able to remove. The top image in FIG. 7 shows a histogram of an image of detection chamber with a high fluorescence signal intensity, the light intensity distribution shifted to the right, representing a high light intensity of collected beads. In contrast, for an image with the lower fluorescence signal intensity (as schematically shown in the bottom image of FIG. 7), the distribution shifts to the left indicating a low light intensity of beads.

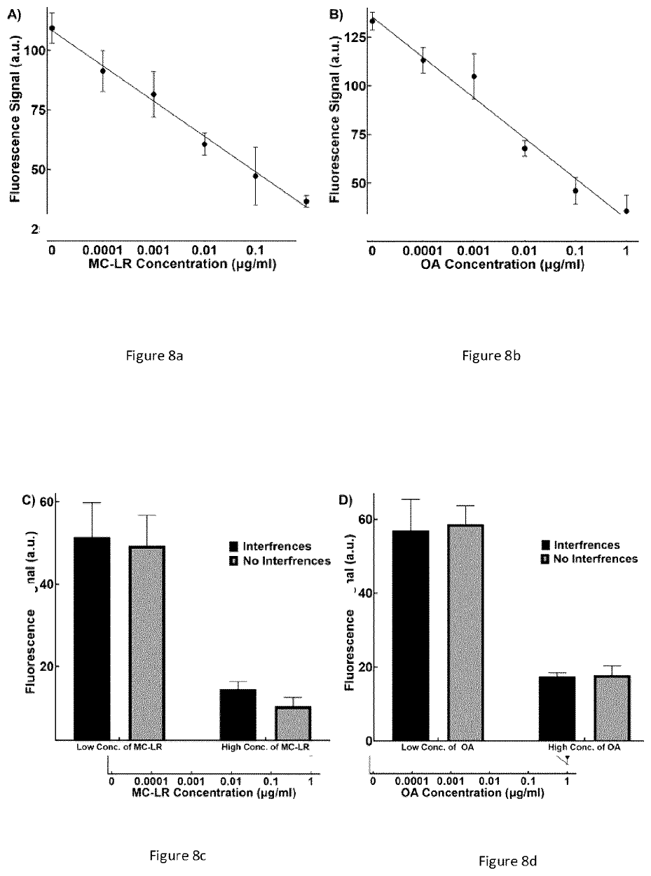

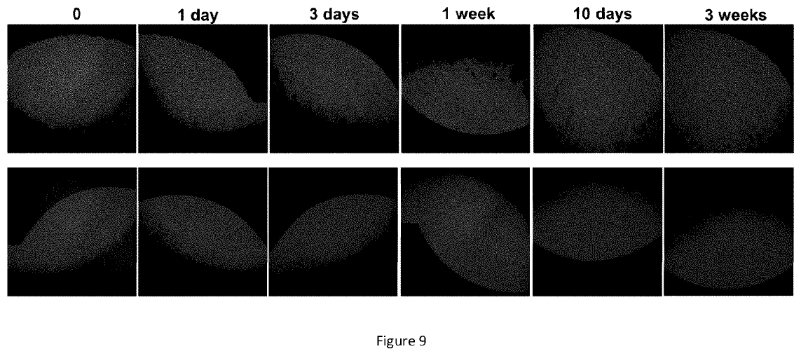

Calibration curves were then generated for MC-LR and OA based on the preferred conditions that were obtained during experiments for mcAb and pA-QD concentrations, mixing device flow rate, the incubation time inside the detection chamber, and the image recognition component. Water samples were spiked with different concentrations of toxin and injected into the microfluidic device (such as taught above). Incubation was performed on the device, and the captured fluorescence images were analyzed using the image recognition component discussed above. A decrease in the fluorescence signals emitted from collected beads by increasing toxin concentrations was observed. Using the device of the disclosure, toxin levels can be measured within 45 mins. Using these measurements, calibration curves were constructed that correlate the fluorescence signal intensity with toxin concentration for MC-LR (FIG. 8a) and OA (FIG. 8b). The device of the disclosure achieved a limit of detection (LOD) of 9.7×10−5 μg/ml and 3.7×10−5 μg/ml for MC-LR and OA detection, respectively.

The selectivity of the device of the disclosure was also tested for specific toxin detection. As water samples are a complex matrix that may include other toxins that can potentially interfere with the detection assay, selectivity of the device toward a target toxin of interest is important in determining the accuracy of the device of the disclosure. The presented immunoassay utilizes antibodies as the recognition element that are highly selective towards the target toxin.

The device of the disclosure was tested for its ability to detect a specific toxin in the presence of interfering species commonly found in freshwaters such as, but not limited to, CYN and STX. These interferences were purposefully selected as CYN is a freshwater toxin similar to MC-LR, and STX is a marine toxin similar to OA. It was observed that the interferences did not affect the assay in a low or high concentration of the target toxin of interest (as shown in FIGS. 8c and 8d), and possible cross-reactivities were avoided, demonstrating high selectivity of the device of the disclosure.

The stability of the bead-bound immunocomplex and pAb-QDs for 0.0001 ug/mL of MC-LR was also tested by monitoring the fluorescence signal of beads over three weeks (as schematically shown in FIG. 9). In this experiment, the beads were retained in the detection chamber and stored in a cold and dark place to measure the fluorescence signal change. It was observed that there was an approximately 12.5%, 23%, and 28.5% drop in the measured signal after 3 days, 10 days, and 3 weeks of storage, respectively. This observation proves the stability of immunocomplex and QDs signal, verifying the robustness of the device of the disclosure.

The capability of the device of the disclosure for simultaneous detection of two toxins was also tested. The multiplexed device was designed and fabricated considering the optimized geometry (such as shown in FIG. 1a or 1b). Defined concentrations of MC-LR and OA were spiked into a sample solution and injected into the device through the sample inlet. The MC-LR and OA reagent solutions were prepared and introduced to the mixing microfluidic device through their specific reagent inlets.

The multiplexity of the device was tested using four samples: 1) a blank solution with no toxins, 2) a MC-LR solution with no OA, 3) an OA solution with no MC-LR, and 4) a solution with both MC-LR and OA. FIG. 1c shows the microscopic images of two detection chambers corresponded to the four conditions. With the introduction of the first solution, a high fluorescence signal was observed in both chambers, which correlates to the zero concentration of toxin. The second and third samples resulted in a reduced fluorescence signal in MC-LR and OA detection chamber, respectively. These results indicate that the reagents are only responsive to their toxin target, confirming no cross-reactivity in the assay. Introducing the fourth solution led to a reduced fluorescence signal in both MC-LR and OA detection chamber. This observation confirms the capability of the device of the disclosure for multiplexed and simultaneous detection of MC-LR and OA in a single run.

Experiments were also performed with respect to the detection of toxins in a spiked lake water sample. Typically, field samples are more complex, and the presence of the interfering substances makes the analysis challenging. For the experiment, the device of the disclosure was employed for the detection of spiked lake water samples. Water samples from Columbia Lake in Waterloo, Ontario, and spiked them with a defined of MC-LR and OA.

| Recovery (%) | 0 μg/ml | 1 μg/ml |
|---|---|---|
| MC-LR | 99.46 | 107.97 |
| OA | 104.43 | 103.88 |

Table 1 shows the toxin recoveries, which were computed as the ratio of the detected concentrations to the spiked concentrations. The recoveries varied from 99% to 108%, demonstrating that the technique can effectively reduce the need for pre-treatment and can be used for real sample analysis. The low matrix effect was owing to the specificity of the antigen-antibody reaction, the large surface area of microbeads, and the amplification of signal by the magnetic collection of beads and the use of QDs as the reporter molecule.

Advantages of the device of the disclosure include, but are not limited to, multiplex capability and accuracy of the assay, as well as portability and cost-effectiveness. The device of disclosure can also be used to monitor toxins and can be modified to detect other toxins in environmental water.

In the preceding description, for purposes of explanation, numerous details are set forth in order to provide a thorough understanding of the embodiments. However, it will be apparent to one skilled in the art that these specific details may not be required. In other instances, well-known structures may be shown in block diagram form in order not to obscure the understanding.

The above-described embodiments are intended to be examples only. Alterations, modifications and variations can be affected to the particular embodiments by those of skill in the art without departing from the scope.

What is claimed is:

1. A method of rapid detection of at least one toxin in a sample using a microfluidic device comprising:
mixing the sample with a selected reagent having metallic beads with functionalized toxins attached to a surface of the metallic beads to generate a mixture solution;
incubating the mixture solution; and
measuring a fluorescence signal of the beads.

2. The method of claim 1 wherein incubating the mixture solution comprises:
incubating the mixture solution with polyclonal antibodies with quantum dots (pAb-QDots) to provide fluorescence to the beads.

3. The method of claim 2 further comprising washing the beads after incubating the mixture solution.

4. The method of claim 1 further comprising before mixing the sample:
dividing the sample into at least two streams.

5. The method of claim 4 wherein mixing the sample with a selected reagent comprises:
mixing each of the at least two streams with a different reagent to generate different mixture solutions, each of the different reagents including metallic beads with functionalized toxins attached to a surface of the metallic beads.

6. The method of claim 1 wherein measuring a fluorescence signal of the beads is performed by flow cytometry, a fluorescence microscope or a plate reader.

7. The method of claim 1 further comprising transmitting the fluorescence signal to a display.

8. A system for rapid detection of at least one toxin in a sample comprising:
a sample receiving area for receiving the sample;
at least one reagent area for receiving a reagent for detection of the at least one toxin in the sample, the reagent including metallic beads with functionalized toxins attached to a surface of the metallic beads; and
at least one detection array in fluid communication with the at least one reagent area and the sample receiving area to generate a mixture solution of the sample;
wherein the at least one detection array includes a mixing module including a herringbone structure.

9. The system of claim 8 wherein the at least one detection array further comprises a detection chamber for receiving the mixture sample.

10. The system of claim 9 further comprising a fluorescence reader.

11. The system of claim 10 wherein the fluorescence reader is a flow cytometry apparatus, a fluorescence microscope or a plate reader.

12. The system of claim 8 further comprising a flow control apparatus for controlling flow rates of the sample and the reagent.

13. The system of claim 8 wherein the sample receiving area further comprises an inlet for receiving the sample.

14. The system of claim 9 further comprising an apparatus for collecting the beads within the detection chamber.

15. A method of reagent production for detection of a toxin of interest in a sample comprising:
combining the toxin of interest with bovine serum albumin (BSA) molecules;
functionalizing the combination of the toxin of interest and the BSA molecules to a surface of a set of metallic beads; and
mixing functionalized metallic beads with an antibody.

16. The method of claim 15 wherein functionalizing the combination is performed by EDC or NHS chemistry.
